(19) United States
(12) Patent Application Publication
(10) Pub. No.: US 2006/0172016 A1
(43) Pub. Date: Aug. 3, 2006

(54) Muscle cramp reliever

(76) Inventors: Vsevolod Kohutiak, Haverford, PA (US); Bohdan Hreschak, Wayne, NJ (US)

Correspondence Address: Eugene E. Renz, Jr., P.C., 205 North Monroe Street, Post Office Box 2056, Media, PA, 19063-9056, US

(21) Appl. No.: 11/127,020

(22) Filed: May 12, 2005

Related U.S. Application Data
(60) Provisional application No. 60/649,075, filed on Feb. 2, 2005.

(51) Int. Cl. A61K 33/14; A61K 36/45

(52) U.S. Cl. 424/680; 424/732

(57) ABSTRACT

A novel beverage composition, which comprises a mixture of water, natural flavorings and color, and a very specific concentration of salts(s), which when administered orally to a person with a muscle spasm, commonly known as a cramp, stops the muscle cramp within a very short period of time, and which period depends on the person's body weight.

This application claims the benefit of U.S. Provisional Application No. 60/649,075, filed Feb. 2, 2005.

### FIELD OF THE INVENTION

The present invention relates generally to water based compositions and more particularly to a water based composition that is formulated for people engaged in physical or other strenuous activity as well as those predisposed to muscle cramps and/or have a history of chronic muscle cramps. More specifically, this invention provides a beverage for consumption by persons needing fast relief for muscle cramps. The invention disclosed herein is directed to a composition which furnishes supplemental electrolytes to the body. The present invention also relates to beverage compositions and beverage concentrates adapted for oral administration of water and physiologically essential lost electrolytes to a human body.

The present invention also relates to carbonated beverage compositions and beverage concentrates adapted for oral administration of water and physiologically essential electrolytes, more particularly, the present invention relates to beverage compositions and beverage concentrates.

Certain endeavors at the formulation of many beverages have not been altogether successful. Moreover, it has been determined that some beverages may be highly detrimental to individuals with certain physical disorders.

Accordingly, the need exists to provide a composition capable of replenishing the necessary electrolytes and specifically depleted essential salts.

### BACKGROUND OF THE INVENTION

A cramp is an involuntary and forcibly contracted muscle that does not relax. Cramps can affect any muscle under a persons voluntary control. Muscles that span two joints are most prone to cramping. Cramps can involve part or all of a muscle, or several muscles in a group. The most commonly affected muscle groups are: back of lower leg/calf, back of thigh (hamstrings), front of thigh (quadriceps).

Cramps in the feet, hands, arms, abdomen and along the rib cage are also very common. Muscle cramps range in intensity from a slight tic to agonizing pain. A cramping muscle may feel hard to the touch and/or appear visibly distorted or twitch beneath the skin. A cramp can last from a few seconds to 15 minutes or longer.

Although the exact cause of muscle cramps is unknown, some researchers believe inadequate stretching and muscle fatigue leads to abnormalities in mechanisms that control muscle contraction. Other factors may also be involved, including exercising or working in intense heat, dehydration and depletion of salt and minerals (electrolytes).

When the body is poorly conditioned one is more likely to experience muscle fatigue, which can alter spinal neural reflex activity. Overexertion depletes a muscle's oxygen supply, leading to build up of waste product and spasm. When a cramp begins, the spinal cord stimulates the muscle to keep contracting.

Heat, dehydration and electrolyte depletion play a critical role. Muscle cramps are more likely when you exercise in hot weather because sweat drains your body's fluids, salt and minerals (i.e., potassium, magnesium and calcium). Loss of these minerals may also cause a muscle to spasm.

Just about everyone will experience a muscle cramp sometime in life. It can happen while you play tennis or golf, bowl, swim or do any exercise. It can also happen while you sit, walk or even just sleep. Sometimes the slightest movement that shortens a muscle can trigger a cramp.

Some people are pre-disposed to muscle cramps and get them regularly with or without any physical exertion. Those at greatest risk for cramps and other ailments related to excess heat include infants and young children, people over age 65, and those who are ill, overweight, overexert during work or exercise, or take drugs or certain medications. Muscle cramps are very common among endurance athletes (i.e., marathon runners and triathletes) and older people who perform strenuous physical activities.

Athletes are more likely to get cramps in the preseason when the body is not conditioned and therefore more subject to fatigue. Cramps often develop near the end of intense or prolonged exercise, or the night after.

Older people are more susceptible to muscle cramps due to normal muscle loss (atrophy) that begins in the mid-40s and accelerates with inactivity. As you age, your muscles cannot work as hard or as quickly as they used to. The body also loses some of its sense of thirst and its ability to sense and respond to changes in temperature.

Physical activity places a great metabolic demand on the human body. One of the important by-products of exercise is increased heat production. Blood flow through exercising muscles may increase to 15 to 20 times the resting level allowing this heat to diffuse from the muscle cell and warm the blood. The exchange of heat between the body and the environment requires that it be transported from the core to the periphery via an augmetation of cutaneous blood flow, by the process of vasodilation. The blood vessels directly below the skin open up and allow blood to circulate near the surface of the body. This acts as a radiation of sorts by bringing the heat to the surface to be dissipated, with the cooler blood returning to the muscles to again be warmed. Heat loss from the skin occurs through conduction, convection (radiation to the environment), or through the evaporation of sweat. During exercise, sweat evaporation is the greatest avenue for heat dissipation.

Loss of sweat from the body is an important physiological consideration. Sweat is hypotonic, that is, it has a lower concentration of salts and other solutes than does blood. Profuse sweating causes excessive loss of body water. This leads to a decrease in sweating rate and less evaporative cooling. Decreased blood volume can lead to a circulatory collapse and decreased evaporative cooling can cause an excessive rise in body temperature. Clinical problems in athletes exposed to environmental heat can vary from temporary heat cramps to fatal heat stroke.

The main problem therefore, in exposure to environmental heat is water loss. The well-hydrated athlete is a more efficient and effective athlete. It is of primary importance that everything be done to provide adequate hydration before, during, and after all sporting events and other types of strenuous activity.

For example, in football, a 250 lb. athlete may lose as much as 10 lb. or 5 quarts of water in a 90 minute practice. Water is a nutrient critical to athletic performance and in certain situations is critical to the athlete's safety. Additionally, strenuous physical activity, such as exercise, particularly on hot days, places a great metabolic demand on the human body. Also, it has been well recognized that muscle cramps of extremities in humans occur as a result of fatigue, dehydration which in part might be as a result of physical activity or other factors to date not yet understood. Once the cramp occurs, which in most instances can be an extremely painful and difficult experience and which is hard to eliminate and requires considerable time and is normally accomplished through massaging the affected part or through the use of topical and oral non-prescription analgesics neither of which have been found to be very effective. Although preparations to prevent muscle cramps have been developed and are widely used in the sports community, the cramps in the muscles of the lower extremities (leg cramps) still occur, which usually puts the affected person into great discomfort. It is being proposed that a composition of only natural ingredients and having a specific concentration or ordinary salt(s) is administered orally to the person affected with the muscle cramp, which within a very short period of time, usually between 15 to 45 seconds eliminates the muscle spasm and the pain associated with it.

Skeletal muscle cramps are one of the most common clinical problems encountered by medical staff who treat athletes at endurance events, especially marathons and triathlons. The lifetime incidence of skeletal muscle cramping in marathon runners and triathletes has been variously reported to be as high as 30% to 50% (3) and 67%. The etiology, diagnosis, and management of this condition have not been definitively elucidated. Muscle cramps can occur in various rare medical conditions, but most athletes who manifest exercise-associated muscle cramping (EAMC) do not have these disorders. The prevalence of EAMC in recreational athletes and differences between rates in elite athletes and recreational athletes are not known.

Also, during exercise in a hot environment, serious deficits in effective circulating volume may occur. Muscular work, independent of environment, results in massive shunting of blood to skeletal muscle, along with a substantial loss of plasma volume into the working muscle. Moreover, effective circulating volume is also diminished by losses of sweat. The deficit in intravascular volume impedes the delivery of heated blood to the periphery for evaporative cooling. Thus, in the dehydrated exercising subject, there is a progressive increase in the core body temperature as sweat losses accumulate. Indeed, salt and water depletion are important predisposing factors to the development of heat-related illnesses.

Exercise is characterized by a marked increase in glucose utilization. The exercising muscle has a greatly increased need for energy. Some of the glucose needed for energy comes from liver glycogen stores. With prolonged exercise, liver glycogen stores are depleted and the rate of glucose production fails to keep pace with glucose utilization, resulting in a fall in the blood glucose concentration. The development of frank hypoglycemia has been described in marathon runners.

Notable among the many physiological responses to physical exertion are increased body temperature, perspiration and pulse rate, a decrease in the blood volume, and biochemical changes associated with the metabolism of compounds to produce energy.

One metabolic change which is associated with continued physical exertion is a shifting of the type of compound used as the primary energy source. In the absence of physical exertion, the metabolism of fat is a primary energy source for the body. During times of exertion, carbohydrates are increasingly used as a source of readily available energy. The body continues to utilize carbohydrates as a major source of energy during prolonged periods of exercise.

If, however, the exercise is particularly strenuous or long in duration, the supply of readily available carbohydrates may become depleted and the body is forced to utilize another source of energy. The metabolism of proteins fills the energy void caused by the depletion of carbohydrates. Unfortunately, the metabolism of protein is not an efficient source of energy for the exercising individual. Protein metabolism results in the utilization of amino acids. This amino acid utilization can result in the depletion of essential amino acids in the plasma. The loss of amino acids can detrimentally affect the person or animal in many ways. One detrimental effect of the depletion of amino acids is a reduction on the body's ability to repair tissue which is damaged in the course of the strenuous exercise.

### OBJECTS OF THE INVENTION

It is an object of this invention to provide a new and improved beverage.

It is another object of this invention to provide a water based beverage which is especially formulated for people engaged in athletic or other strenuous activity.

It is still another object of this invention to provide a water based beverage which is especially formulated to prevent cramps in people engaged in strenuous activity.

It is a further object of this invention to provide a water based beverage which includes electrolytes normally lost in strenuous activity.

It is still a further object of this invention to provide a water based beverage useful for treating cramps which includes electrolytes normally lost in strenuous activity and in the amounts in which said electrolytes are normally lost.

It is yet another object of this invention to stop within seconds the spasm commonly known as the cramp of the muscle usually in the lower extremity (leg cramp), but not limiting it to the muscle complex of the lower extremity.

It is still an object of this invention to provide a new and improved beverage for stopping cramps.

It is also an object of the present invention to provide a beverage composition that contains essential physiological electrolytes.

It is yet a further object of the present invention to provide a beverage for supplementing the requirements of essential salts in a palatable and highly efficient form.

### SUMMARY OF THE INVENTION

The present invention provides a beverage for the rapid relief of muscle cramps, comprising by weight: (a) 2.0 to 8.0% of a sweetener selected from the group consisting of fructose, glucose and high fructose corn syrup solids or mixtures thereof; (b) 1.0 to 5.0% of sodium chloride commonly known as table salt. The beverage cal also include a salt selected from the group consisting of sodium citrate, potassium chloride, potassium citrate or mixtures thereof; (c) 5.0 to 9.0% of a fruit juice; (d) 1.0 to 3.0% of a fruit concentrate; (e) optionally 0.2 to 0.8% of an edible acceptable acid; and (f) the balance being water.

The instant invention also provides a beverage useful for stopping within seconds the spasm commonly known as the cramp of the muscle usually in the lower extremity (leg cramp), but not limiting it to the muscle complex of the lower extremity. Briefly stated, the present invention relates to a novel composition, which comprises the preparation of solids dissolved in water with a very specific concentration of salt(s), which when administered to a person undergoing muscle spasm/cramp, eliminates the cramp usually within 15 to 45 seconds, and which time depends primarily on that person's body weight. In spite of all the efforts to contain the muscular spasm once it develops, there has been no preparation known in the prior art, which works in such a rapid manner and which totally eliminates the pain and discomfort of the spasm.

### DESCRIPTION OF THE PREFERRED EMBODIMENTS

The present invention relates to a beverage useful for relieving cramps and supplementing the requirements of essential salts in a mammalian body and, especially, a human body, where these components are depletable through vigorous physical activity. For example, an athlete engaged in strenuous activity requires a ready source of energy for endurance, and replacement of both body fluids and essential salts lost through perspiration. Likewise, individuals working in a hot, humid atmosphere have similar requirements to maintain efficiency and productivity.

The term “beverage” as used herein denotes a composition that is single-strength and ready to drink, that is, drinkable. As used herein, “beverage concentrate” refers to a concentrate that is either in liquid form or in essentially dry mixture form. The essentially dry mixture can be in the form of a powder. The concentrate is usually formulated to provide a drinkable beverage composition or a final beverage when reconstituted or diluted with water, either carbonated or non-carbonated.

The present invention also provides a novel beverage and methods for providing rapid relief of cramps resulting from the adverse physiological effects associated with physical exertion and heat exposure or simply inactivity as during sleeping. The instant invention can be used with humans and other animals.

Accordingly, the instant invention provides a novel beverage comprising: (a) water; (b) electrolytic compound(s); (c) a sugar; (d) a fruit juice; (e) a fruit concentrate and (f) optionally a food acceptable edible acid.

In the practice of this invention, ordinary table salt (sodium chloride) is the preferred component, however other salts such as potassium chloride, sodium citrate, potassium citrate and mixtures thereof may be used. The preferred range of concentration for the salt component of the invention is 1.0 to 5.0% by weight with the most preferred range being 2.5 to 3.5% by weight.

The sweetener of the invention is typically high fructose corn syrup solids which makes the oral intake of the composition more palatable, however glucose, fructose and other sweeteners may be used alone or in combination. The preferred range for the sweetener is 5.0 to 8.0% by weight with the most preferred range being 2.0 to 10.0% by weight.

Additional carbohydrate sweeteners in the practice of the instant invention includes monosaccharides, oligosaccharides, complex polysaccharides, or mixtures thereof. The monosaccharides, include tetroses, pentoses, hexoses, and ketohexoses. Examples of hexoses are aldohexoses such as glucose and dextrose, known as grape sugar. The amount of dextrose used for the drinkable beverage composition of the present invention preferably is in a range of from about 1 to about 4 weight percent, based on the total weight of the beverage composition, and more preferably from about 1.5 to about 2 weight percent of the total weight of the beverage composition. Fructose, known as fruit sugar, is a ketohexose. Preferably, the range of fructose present in the present drinkable beverage composition is from about 1 to about 5 weight percent of the total weight of the beverage composition, and more preferably from about 1 to about 3 weight percent of the total weight of the beverage composition. For the essentially dry mixture suitable for constituting with water to form a final aqueous beverage, the preferable range of crystalline fructose is from about 1 to about 5 weight percent, based on the total weight of the final aqueous beverage, and more preferably in the range of from about 1 to about 3 weight percent of the total weight of the final aqueous beverage. Another important class of oligosaccharide is a disaccharide. An example of a disaccharide that can be used in the beverage of the invention is sucrose, also known as cane sugar or beet sugar. One of the complex polysaccharides usable for the present invention is maltodextrin. Maltodextrins are a form of complex carbohydrate molecule several glucose units in length. They are spray-dried carbohydrate ingredients made by controlled hydrolysis of corn starch.

The Food and Drug Administration defines maltodextrin as a nonsweet, nutritive saccharide polymer that consists of D-glucose units linked primarily by alpha-1,4-bonds. The preferred range of maltodextrin for the drinkable beverage composition is between about 2 to about 7 weight percent of the total weight of the drinkable beverage composition, and more preferably from about 3 to about 4 weight percent, based on the total weight of the drinkable beverage composition.

Additionally, low-caloric sweeteners can be used in the practice of the invention. Examples of such non-caloric or low-caloric carbohydrate sweeteners include, but are not limited to, saccharin, cyclamates, acetosulfam, sorbitol, xylitol, L-aspartyl-L-phenyl-alanine ester, (e.g. aspartame), L-aspartyl-D-alanine alkyl amides, L-aspartyl-L-1-hydroxymethylalkaneamide, L-aspartyl-1-hydroxyethylalkaneamide, and the like. The amount of the non-caloric sweetener used depends on the particular sweetener, or mixture of sweeteners, and the sweetness intensity desired. Generally, the non-caloric or low-caloric sweetener ranges from about 0.5 to about 14 weight percent, based on the total weight of the drinkable beverage composition. In diet drinks, if desired, one percent weight of fructose, based on the total weight of the drinkable beverage composition, can be used.

The flavoring agents of the invention is a fruit juice such as cranberry syrup or its concentrate however other fruit or non fruit type flavors and their derivatives and or bittering agents may be used. Other fruit flavors include orange juice, papaya juice, pineapple juice, apple juice, grape juice, apricot, pear, tomato and many others known in nature. Synthetic flavorants may also be used in the practice of the instant invention. The fruit juice is present in the range of 5.0% to 9% by weight although higher amounts may be used to provide proper flavoring and palatability. The adjustments necessary to provide consumer acceptability are well within an individual of ordinary skills in the art of beverages.

The beverage composition and the concentrate of the present invention further includes a flavor selected from fruit flavors, botanical flavors, and mixtures thereof. As used herein, the term “fruit flavor” denotes flavors derived from the natural edible reproductive parts of seed plants as well as synthetically prepared flavors made to simulate fruit flavors derived from natural sources. Preferred flavoring additives are citrus flavors, such as orange flavor, lemon flavor, lime flavor, fruit punch, and mixtures thereof. Other flavors, such as grape flavor, cherry flavor, apple flavor, and mixtures thereof, can also be used. The amount of soluble flavor additive used depends upon the flavor or flavors selected, the flavor impression desired, and the form of flavor additive used. When a concentrated flavor is used as an additive, the amount of flavor additive can vary from about 0.05 to about 1.5 weight percent based on the total weight of the drinkable beverage composition.

The fruit concentrate is in the range of 1% to 3% by weight. As with the fruit juices mentioned above, the fruit concentrate may be derived from the same juices.

The edible acid in the composition is typically present in a concentration range of about 0.2% to 0.8% by weight. The edible acid component of the present invention can be selected from phosphoric acid, citric acid, malic acid, succinic acid, tartaric acid, fumaric acid, and mixture thereof. In the case of malic acid and succinic acid, they can be wholly or partly substituted thereof. The acids can be in their undissociated forms or as their respective “sour salts,” such as phosphate, dihydrogen phosphate, citrate, malate, or the combination thereof. Preferably, the pH range of the drinkable beverage composition is from about 2 to about 6.5, and more preferably from about 3 to about 5.

The phosphate and the citrate ions present in the beverage compositions or concentrates can also function as a buffer system to maintain the drinkable beverage composition within a pH range.

The beverage of the invention may optionally contain affective amounts of ascorbic acid to equalize the composition and help the shelf life of the composition. However, the amount added may be up to a level that provides 200 mg of ascorbic acid per 16 fluid ounces of beverage.

The beverage may also optionally contain additional vitamins which are beneficial to the human body. For example vitamin E and al the B vitamins may be incorporated into the beverage of the instant invention in amounts that do not substantially affect the final flavor of the beverage.

In the practice of this invention, water was used as a principal solvent but should not be a limiting factor, since the novel composition of ingredients as described in 2, 3 and 4 can be taken orally without water as a solvent and then followed by drinking water to wash the novel composition down. In order to further illustrate the present invention, but in no way to limit it, the following examples are provided which were used by both inventors and a number of people afflicted by muscle cramps.

The drinkable beverage composition may be prepared by mixing together all of the ingredients. The mixture is then dissolved in water and agitated with a mechanical stirrer until all of the ingredients have gone into solution. It may be necessary for the solution to be heated to a temperature between about 50° C. and about 100° C. to get all the ingredients dissolved. The preservative i.e., ascorbic acid can then be added. After the mixture has been adjusted to a desired pH with an acid component, the beverage may be bottled, capped, and pasteurized at about 75° C. for about 20 minutes.

One way to prepare the concentrate form of the liquid beverage would be to start with less than the required volume of the liquid solvent that is used in the preparation of the drinkable beverage composition. Another way would be to partially dehydrate the finally prepared drinkable beverage composition to remove only a portion of the liquid solvent and any other volatile liquids present. Dehydration can be accomplished in accordance with a well known procedure, such as evaporation under vacuum. The concentrate can be in the form of a relatively thick, syrupy liquid or a solid. The solid can be in the form of an essentially dry powder or a tablet. The concentrate can later be reconstituted with a proper amount of water to make the final drinkable beverage composition.

Carbon dioxide can be introduced either into the water to be mixed with the beverage concentrate, or into the drinkable beverage composition, to achieve carbonation. The carbonated beverage composition can then be stored in a container, such as a bottle or a can and then sealed. The amount of carbon dioxide introduced into the beverage composition depends upon the particular flavor system used and the amount of carbonation desired. Usually, carbonated beverages of the present invention contain from about 1.0 to about 4.5 volumes of carbon dioxide. Preferred carbonated beverages contain from about 2 to about 3.5 volumes of carbon dioxide.

The essentially dry mixture of the beverage composition can be prepared by blending the proper amounts and ratios of all the required dry ingredients together. Alternatively, the finally prepared drinkable beverage composition can be dehydrated to give the essentially dry mixture of the beverage composition. An edible binding agent, such as starch, can be added to a powder form of the beverage composition and the resulting mixture compacted into tablets. The essentially dry mixture, either as powder, granules or tablets, can later be dissolved in a proper amount of water, carbonated or non-carbonated, to make the final drinkable beverage composition.

The ratio of ingredients in the composition may also be adjusted for changing environmental or physiological conditions. For example, in cold weather, the composition may contain a lower concentration of electrolytes. Also, for individuals who desire a lower calorie drink, the sugar may be replaced with an artificial sweetener such as aspartame or saccharin.

The beverage of the present invention may be compounded with water and suitably bottled and stored. Alternatively, all of the components, save for water, may be prepared in advance as a concentrate for ease of handling and transportation.

Another important aspect of the present invention is to provide a beverage that stops cramps in a period of about 15-40 seconds.

The following are examples which illustrate materials, methods and procedures, including the best mode, for practicing the invention. These examples are illustrative and should not be construed as limiting. Where concentrations are expressed as percentages, these percentages refer to weight ratios.

### EXAMPLE I

In a steam injected double wall vessel of 15 quart capacity, equipped with a high speed mixer were placed 7.85 pounds of water, 0.80 pounds of high fructose corn syrup solids (HFCSS), 0.80 pounds of cranberry juice, 0.30 pounds of salt, 0.20 pounds of cranberry juice concentrate, and 0.05 pounds of citric acid. The ingredients were blended by mixing 5 minutes with a high speed mixer at 50 revolutions per minute. Steam was introduced into the wall of the vessel and the mixture under reduced agitation of 25 revolutions per minute was brought to a boil. It was allowed to cool by stopping the steam and introducing cold water into the vessel wall. The resulting beverage was packed under vacuum into 8 ounces glass jars and capped.

### EXAMPLE II

Using the procedure of Example 1, a similar composition was prepared from 7.85 pounds of water, 0.70 pounds of fructose corn syrup solids, 0.80 pounds of cranberry juice, 0.30 pounds of salt, 0.20 pounds of cranberry juice concentrate, 0.10 pounds of potassium citrate and 0.05 pounds of citric acid without affecting the essential properties of the finished product.

### EXAMPLE III

Using the procedure of Example 1, a similar composition was prepared from 7.85 pounds of water, 0.70 pounds of fructose corn syrup solids, 0.80 pounds of lime juice, 0.30 pounds of salt, 0.20 pounds of lime juice concentrate, 0.10 pounds of potassium citrate and 0.05 pounds of citric acid without affecting the essential properties of the finished product.

### EXAMPLE IV

In a steam injected double wall vessel of 15 quart capacity, equipped with a high speed mixer were placed 7.85 pounds of water, 0.80 pounds of high fructose corn syrup solids (HFCSS), 0.80 pounds of apple juice, 0.30 pounds of salt, 0.20 pounds of apple juice concentrate, and 0.05 pounds of citric acid. The ingredients were blended by mixing 5 minutes with a high speed mixer at 50 revolutions per minute. Steam was introduced into the wall of the vessel and the mixture under reduced agitation of 25 revolutions per minute was brought to a boil. It was allowed to cool by stopping the steam and introducing cold water into the vessel wall. The resulting beverage was packed under vacuum into 8 ounces glass jars and capped.

### EXAMPLE V

In a steam injected double wall vessel of 15 quart capacity, equipped with a high speed mixer were placed 7.85 pounds of water, 0.80 pounds of high fructose corn syrup solids (HFCSS), 0.80 pounds of apricot juice, 0.30 pounds of salt, 0.20 pounds of apricot juice concentrate, and 0.05 pounds of citric acid. The ingredients were blended by mixing 5 minutes with a high speed mixer at 50 revolutions per minute. Steam was introduced into the wall of the vessel and the mixture under reduced agitation of 25 revolutions per minute was brought to a boil. It was allowed to cool by stopping the steam and introducing cold water into the vessel wall. The resulting beverage was packed under vacuum into 8 ounces glass jars and capped.

### EXAMPLE VI

In a house hold type blender/mixer a composition comprised of water, high fructose corn syrup solids, white grape juice, pear juice, salt (3% by weight), natural lemon juice and critic acid was blended together for thirty seconds, transferred to a pan and brought to a quick boil. Hot blend was allowed to cool to 170-175° Fahrenheit and vacuum packed. The finished blend was quite palatable and had a pleasant taste. It was used approximately 6 months later by one of the inventors during the night when the inner upper leg muscle was struck by a cramp. A serving of 2.5 ounces of the novel composition stopped the pain and discomfort in exactly 26 seconds.

### EXAMPLE VII

In a house hold type blender/mixer a composition comprised of water, cranberry juice, high fructose corn syrup solids, salt (3% by weight), cranberry juice concentrate and citric acid was blended together for thirty seconds, transferred to a pan and brought to a quick boil. Hot mixture was allowed to cool to 170-175° Fahrenheit and packed in glass jars. The finished blend had a tart taste but was quite palatable. It was vacuum packed and used by second inventor number of times on the tennis court where he so often is bothered with the leg cramp. On the average it took between 17 and 25 seconds to eliminate the spasm and the pain completely. The serving size was 2 ounces.

### EXAMPLE VIII

In a house hold type blender/mixer a composition comprised of water, cranberry juice, high fructose corn syrup solids, salt (2.5% by weight) cranberry juice concentrate, potassium citrate, potassium phosphate and ascorbic acid was blended together for thirty seconds, transferred to a pan and brought to a quick boil. It was cooled to 170-175° Fahrenheit. The finished blend had tart taste but was quite palatable. It was vacuum packed and used on several occasions by various people with muscle cramps. The record show that it took on the average between 15 and 35 seconds to eliminate the muscle cramp and the discomfort of the pain associated with it.

### EXAMPLE IX

The optimal rate of administration of the novel composition described here can depend upon the physiological characteristics of the individual receiving the fluid, the nature of the physical exertion or exposure, and the environmental conditions.

The ratio of ingredients in the composition may also be adjusted for changing environmental or physiological conditions. Also, for individuals who desire a lower calorie drink, the sugar may be replaced with an artifical sweetener such as aspartame or saccharin. For individuals who are concerned about high blood pressure, the drink can contain reduced concentrations of sodium.

### EXAMPLE X

The composition of the subject invention may also be prepared in a dehydrated, powder, or concentrate form for convenience of sale or shipment. When formulated in this way, the product could be reconstituted by the addition of water. The preparation of such a product in the dehydrated, powder, or concentrate form is well known to those skilled in the art. See for example, U.S. Pat. Nos. 4,042,684 and 4,322,407.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed and desired to be secured by Letters Patent is:

1. A beverage for the rapid relief of muscle cramps, comprising by weight:
(a) 2.0 to 10.0% of a sweetener selected from the group consisting of fructose, glucose and high fructose corn syrup solids or mixtures thereof;
(b) 1.0 to 5.0% of a salt selected from the group consisting of sodium chloride, sodium citrate, potassium chloride, potassium citrate or mixtures thereof;
(c) 5.0 to 9.0% of a fruit juice;
(d) 1.0 to 3.0% of a fruit concentrate;
(e) optionally 0.2 to 0.8% of an edible acceptable acid; and
(f) the balance being water.

2. The beverage of claim 1 wherein said sweetener is fructose.

3. The beverage of claim 1 wherein said sweetener is glucose.

4. The beverage of claim 1 wherein said sweetener is high fructose corn syrup solids.

5. The beverage of claim 4 wherein said fruit juice is cranberry juice.

6. The beverage of claim 5 wherein said salt is sodium chloride.

7. The beverage of claim 6 wherein said fruit concentrate is cranberry concentrate.

8. The beverage of claim 7 wherein said food edible acid is citric acid.

9. The beverage composition of claim 1 further comprising Vitamin C.

10. The beverage composition of claim 9 wherein said Vitamin C is in an amount ranging from about 150 to about 2000 mg per liter of said beverage composition.

11. The beverage composition of claim 1 further comprising Vitamin E.

12. The beverage composition of claim 11 wherein said Vitamin E is in an amount ranging from about 20 to about 60 I.U. per liter of said beverage composition.

13. The beverage composition of claim 1 further comprising an edible buffering component.

14. The beverage composition of claim 13 wherein said edible buffering component is selected from a group consisting of bicarbonate ions and phosphate ions.

15. The beverage composition of claim 1 further comprising a flavoring additive.

16. The beverage composition of claim 15 wherein said flavoring additive is present in an amount ranging from about 0.01 to about 1 weight percent based on the total weight of said beverage composition.

17. A method for relieving muscle cramping which method comprises drinking the beverage of claim 1, thus treating said muscle cramps.
